(19) United States
(12) Patent Application Publication
(10) Pub. No.: US 2016/0100789 A1
(43) Pub. Date: Apr. 14, 2016

(54) COMPUTER-AIDED DIAGNOSIS SYSTEM AND COMPUTER-AIDED DIAGNOSIS METHOD

(71) Applicant: National Central University, Jhongli City (TW)

(72) Inventor: Hui-Yang Huang, Taipei City (TW)

(21) Appl. No.: 14/512,558

(22) Filed: Oct. 13, 2014

(51) Int. Cl. A61B 5/00; A61B 5/103

(52) U.S. Cl. CPC A61B 5/444; A61B 5/1032; A61B 5/0077; A61B 5/7282; A61B 5/7246; A61B 5/7267; A61B 2560/0475; A61B 2576/02

(57) ABSTRACT

Disclosed herein is a computer-aided diagnosis system and a computer-aided diagnosis method. This method includes the step of performing a principal component analysis to acquire an effect of light and shade portions from a color image of skin to serves as a first principal component. This method performs the principal component analysis further to acquire a second principal component and a third principal component, and the second and third principal components contain color variability. The third principal component is correlated with a skin cancer and serves as a main indicator of variegated colors for malignancy diagnosis.

### BACKGROUND

1. Field of Invention

The present invention relates to computer systems. More particularly, the present invention relates to computer-aided diagnosis systems.

2. Description of Related Art

Skin cancer is a commonly occurring malignancy in fair-skinned populations. In the last decade, the number of skin cancer treatments grew substantially, and the cost of skin cancer management was among the highest of all cancers in the United States. There were approximately 76,250 new cases of melanoma and approximately 8,790 new melanoma-related deaths in 2012 in the United States. Although the incidence rates of melanoma in Asians are lower than in Caucasians, nonmelanoma skin cancers, such as squamous cell carcinoma (SCC) or basal cell carcinoma (BCC), contribute to significant morbidities among fairer-skinned Asians. In a recent estimation by the Australian government, the total cost of diagnosing and treating non-melanoma skin cancer was 511 million Australian dollars in 2010 and will be 703 million in 2015.

It is always important for clinicians to be able to recognize and accurately diagnose skin cancer in its early stages. When conducting a skin cancer screening, doctors usually identify suspect lesions by visual examination, which is highly dependent on specific training, and diagnostic accuracy can vary greatly among individuals with varied experiences. In the U.K. and Australia, there has been increasing interest in improving the diagnostic performance of general practitioners in recognizing and accurately diagnosing skin cancers. With the development of computer-aided image analysis technologies, physicians may obtain an objective “second opinion” from computer-aided detection (CAD) or computer-aided diagnosis (CADx) software to refine their diagnoses. In clinical practice, CAD has been widely used in the field of lesion detection, such as breast lesion detection in mammography, lung nodule detection on chest radiographs or CT scans, and polyp detection in CT colonography. CADx has also been applied to the analysis of nuclear medicine images, skin lesions, and histopathological images. CADx has been demonstrated to increase the diagnostic accuracy of trainees in the field of radiology. In dermatology, the benefits of the integration of CADx into the clinical diagnosis of pigmented skin lesions for dermatologists remain under investigation.

It has been suggested that the accuracy rate of clinicians can be improved with the support of dermatoscopy. However, this approach depends on specific training of a limited population of clinicians, and mainly dermatologic specialists who manage skin tumors. Moreover, previous CADx studies in dermatology based on digitized color images or dermatoscopic images mainly focused on melanoma or melanocytic skin cancer detection. This approach is not generally applicable, especially given the low incidence of melanoma in Asians. We became interested in developing a diagnostic system that can also classify non-melanocytic skin cancers in Asian people. Considering easy accessibility to digital photography, the ability to analyze regular digital photographic images would be invaluable for general practitioners. This method could possibly play an important role in the remote analysis of skin lesions using digital photography for hospitals lacking dermatologic specialists.

### SUMMARY

The following presents a simplified summary of the disclosure in order to provide a basic understanding to the reader. This summary is not an extensive overview of the disclosure and it does not identify key/critical components of the present invention or delineate the scope of the present invention. Its sole purpose is to present some concepts disclosed herein in a simplified form as a prelude to the more detailed description that is presented later.

In one aspect, the purpose of the present disclosure is to investigate the potential for skin lesion classification by CADx utilizing regular digital photographic images. In particular, present disclosure aimed to develop new color-related features for conventional photography by investigating multicolor channel characteristics using Pearson correlation coefficients and principal component analysis (PCA).

In one embodiment, a computer-aided diagnosis system comprises a processor and a memory. The processor is capable of executing one or more computer executable instructions. The memory comprises a computer program executable by the processor, the computer program which, when executed by the processor: performing a principal component analysis to acquire an effect of light and shade portions from a color image of skin to serves as a first principal component.

In one embodiment, the principal component analysis further analyzes a second principal component and a third principal component, and the second and third principal components contain color variability.

In one embodiment, the third principal component is correlated with a skin cancer and serves as a main indicator of variegated colors.

In one embodiment, the processor acquires a two-dimensional correlation coefficient from the color images, the two-dimensional correlation coefficient is different from the principal component analysis and is computed by machine learning to enhance an accuracy of malignancy index of the variegated colors.

In one embodiment, the processor acquires one of more one-dimensional statistical parameters from the color images, the one-dimensional statistical parameters including a variance parameter, an entropy parameter and a skewness parameter are different from the principal component analysis and are computed by machine learning to enhance the accuracy of the malignancy index of the variegated colors.

In one embodiment, the computer-aided diagnosis system further comprises an image-capturing device. The image-capturing device is configured to capture the color image of the skin.

In one embodiment, the image-capturing device is a camera.

In one embodiment, the color image includes a lesion and a normal skin portion surrounding the lesion to improve a stability of the malignancy index of the variegated colors.

In one embodiment, the processor is configured to diagnose a skin cancer based on the first, second and third principal components, the two-dimensional correlation coefficient and one-dimensional statistical parameter.

In one embodiment, the principal component analysis is applied in a RGB color model.

In one embodiment, a computer-aided diagnosis method comprises the step of performing a principal component analysis to acquire an effect of light and shade portions from a color image of skin to serves as a first principal component.

In one embodiment, the principal component analysis further analyzes a second principal component and a third principal component, and the second and third principal components contain color variability.

In one embodiment, the third principal component is correlated with a skin cancer and serves as a main indicator of variegated colors.

In one embodiment, the computer-aided diagnosis method further acquires a two-dimensional correlation coefficient from the color images, the two-dimensional correlation coefficient is different from the principal component analysis and is computed by machine learning to enhance an accuracy of malignancy index of the variegated colors.

In one embodiment, the computer-aided diagnosis method further acquires one of more one-dimensional statistical parameters from the color images, the one-dimensional statistical parameters including a variance parameter, an entropy parameter and a skewness parameter are different from the principal component analysis and are computed by machine learning to enhance the accuracy of the malignancy index of the variegated colors.

In one embodiment, the computer-aided diagnosis method further captures the color image of the skin by an image-capturing device.

In one embodiment, the image-capturing device is a camera.

In one embodiment, the color image includes a lesion and a normal skin portion surrounding the lesion to improve a stability of the malignancy index of the variegated colors.

In one embodiment, the computer-aided diagnosis method further diagnoses a skin cancer based on the first, second and third principal components, the two-dimensional correlation coefficient and one-dimensional statistical parameter.

In one embodiment, the principal component analysis is applied in a RGB color model.

In view of the foregoing, the technical solutions of the present disclosure result in significant advantages and beneficial effects, when compared with conventional methods. The implementation of the above-mentioned technical solutions achieves substantial technical improvement and provides utility that is widely applicable in the industry.

Many of the attendant features will be more readily appreciated, as the same becomes better understood by reference to the following detailed description considered in connection with the accompanying drawings.

### BRIEF DESCRIPTION OF THE DRAWINGS

The present description will be better understood from the following detailed description read in light of the accompanying drawing, wherein:

FIG. 1 is a block diagram of a computer-aided diagnosis system according to one exemplary embodiment of the present disclosure;

FIG. 2 shows the skin lesion image pixels in the RGB color space according to one exemplary embodiment of the present disclosure;

FIG. 3 shows a histogram of a benign skin tumor and a histogram of a normal skin portion surrounding the benign skin tumor according to one exemplary embodiment of the present disclosure, in which pixels are projected on the first principal axis;

FIG. 4 shows a histogram of a malignant skin tumor and a histogram of a normal skin portion surrounding the malignant skin tumor according to another exemplary embodiment of the present disclosure, in which pixels are projected on the first principal axis;

FIG. 5 shows a histogram of a benign skin tumor and a histogram of an normal skin portion surrounding the benign skin tumor according to one exemplary embodiment of the present disclosure, in which pixels are projected on the second principal axis;

FIG. 6 shows a histogram of a malignant skin tumor and a histogram of a normal skin portion surrounding the malignant skin tumor according to another exemplary embodiment of the present disclosure, in which pixels are projected on the second principal axis;

FIG. 7 shows a histogram of a benign skin tumor and a histogram of a normal skin portion surrounding the benign skin tumor according to one exemplary embodiment of the present disclosure, in which pixels are projected on the third principal axis;

FIG. 8 shows a histogram of a malignant skin tumor and a histogram of a normal skin portion surrounding the malignant skin tumor according to another exemplary embodiment of the present disclosure, in which pixels are projected on the third principal axis;

FIG. 9 illustrates the correlation of red-, green-channel pixel values for a skin lesion image according to one exemplary embodiment of the present disclosure.

### DETAILED DESCRIPTION

In the following detailed description, for purposes of explanation, numerous specific details are set forth in order to attain a thorough understanding of the disclosed embodiments. In accordance with common practice, the various described features/elements are not drawn to scale but instead are drawn to best illustrate specific features/elements relevant to the present invention. Also, like reference numerals and designations in the various drawings are used to indicate like elements/parts. Moreover, well-known structures and devices are schematically shown in order to simplify the drawing and to avoid unnecessary limitation to the claimed invention.

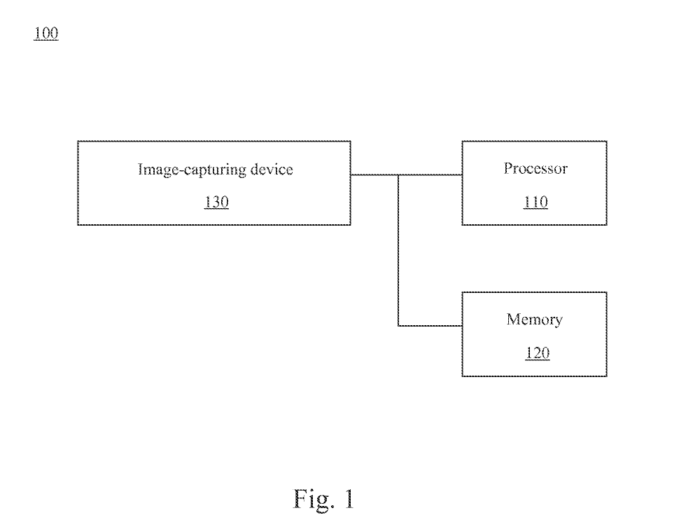

FIG. 1 is a block diagram of a computer-aided diagnosis system 100 according to one exemplary embodiment of the present disclosure. As shown in FIG. 1, the computer-aided diagnosis system 100 comprises a processor 110 (e.g., CPU) and a memory 120 (e.g., RAM or ROM). The processor 110 is capable of executing one or more computer executable instructions. The memory 120 comprises a computer program executable by the processor 110, the computer program is executed by the processor 110 to perform a computer-aided diagnosis method. The computer-aided diagnosis method includes the step of performing a principal component analysis to acquire an effect of light and shade portions from a color image of skin to serves as a first principal component (PC1), where the principal component analysis is applied in a RGB color model or the like.

In one embodiment, the principal component analysis further analyzes a second principal component (PC2) and a third principal component (PC3), and the second and third principal components contain color variability. The third principal component is correlated with a skin cancer and serves as a main indicator of variegated colors.

In one embodiment, the processor 110 acquires a two-dimensional correlation coefficient from the color images, the two-dimensional correlation coefficient is different from the principal component analysis and is computed by machine learning to enhance an accuracy of malignancy index of the variegated colors.

In one embodiment, the processor 110 acquires one of more one-dimensional statistical parameters from the color images, the one-dimensional statistical parameters including a variance parameter, an entropy parameter and a skewness parameter are different from the principal component analysis and are computed by machine learning to enhance the accuracy of the malignancy index of the variegated colors.

In FIG. 1, the computer-aided diagnosis system 100 further comprises an image-capturing device 130. The image-capturing device (e.g., a camera) is configured to capture the color image of the skin. In one embodiment, the color image includes a lesion and a normal skin portion surrounding the lesion to improve a stability of the malignancy index of the variegated colors.

In use, the processor 110 is configured to diagnose a skin cancer based on the first, second and third principal components, the two-dimensional correlation coefficient and one-dimensional statistical parameter.

The software system (i.e., above computer program) includes three independent computational elements: skin lesion segmentation, a feature extraction, and machine learning.

First, the skin lesion segmentation is a collection of photos of skin lesions with known diagnoses. The lesion area of each photo is segmented by means of using the feature extraction. The feature extraction can use a fully automatic algorithm or a manually operated graphical user interface to segment skin lesions. In one of experiment, a dermatologist to assure accurate segmentation for training the system uses manual segmentation. The memory 120 (shown FIG. 1) or the like stores the segmentation results.

The feature extraction is the main function executed by the processor 110 for extracting features from the photos (e.g., color image of skin) and the segmentation information for evaluating and quantizing the malignancy according to the ABCD rules (i.e., asymmetry (A), border (B), color (C), and diameter (D)) and other factors such as surface textures.

The feature extraction can extract shape features such as asymmetry index, compactness, roundness, and radial variance. Asymmetry index and roundness are useful features for evaluating a lesion's asymmetry (the A-rule in the ABCE rules). Compactness and radial variance are good features for evaluating a lesion's border irregularity (the B-rule in the ABCD rules).

The feature extraction can extract conventional texture features such as Tamura's coarseness and gray-level-run-length-matrix (GLRLM). Additionally or alternatively, the feature extraction can extract extracting color features such as variance, entropy, skewness and two new groups of color features: the principal components (PC) using principal component analysis (PCA) and Pearson product-moment correlation coefficients (Corr).

Above color feature extraction is the main invention. The color feature extraction can extract lesion's RGB pixel values, where Var( ) stands for variance, which is a statistical measure of the spread of a dataset. Var(R), Var(G), Var(B), Var(X) stand for variances of red-, green-, blue-channels, brightness of the examined lesion's pixels. The Var( ) algorithm is applied to the segmented lesion region only. As to improve computational stability in deriving the malignancy index, the Var( ) algorithm is also applied to the lesion region plus some neighboring normal skin pixels, which serve as color references. Similarly, Ent( ) stands for entropy, which is a statistical measure of randomness, and Skw( ) stands for skewness, which is a measure of distribution asymmetry. Ent( ) and Skw( ) are applied to the images in the same way as Var( ). Because Var( ), Ent( ), and Skw( ) are applied to a single variable of red, green, blue, or brightness. Therefore, they are called one-dimensional color features (i.e., one-dimensional statistical parameters) in this description.

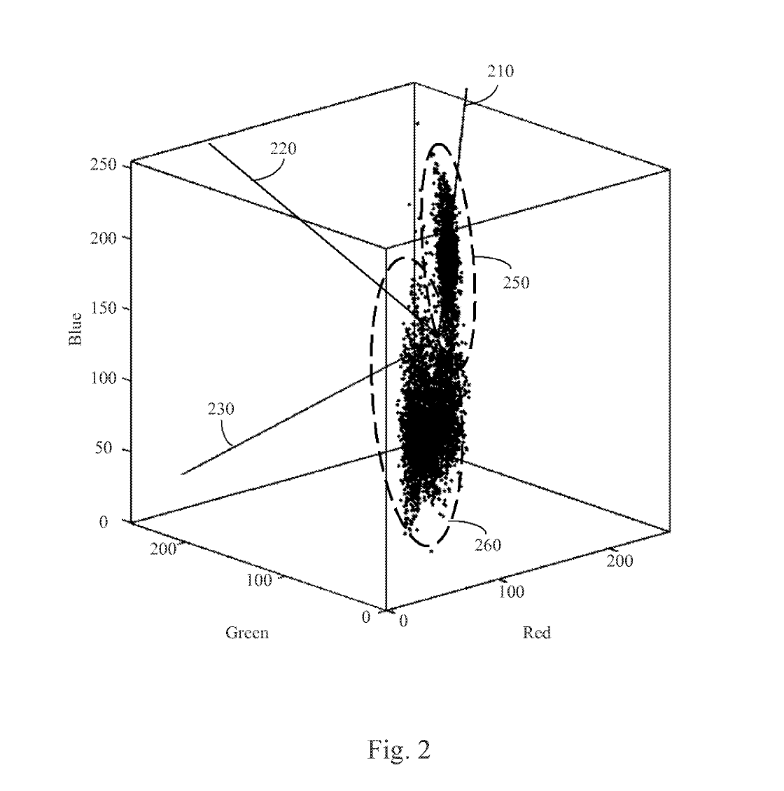

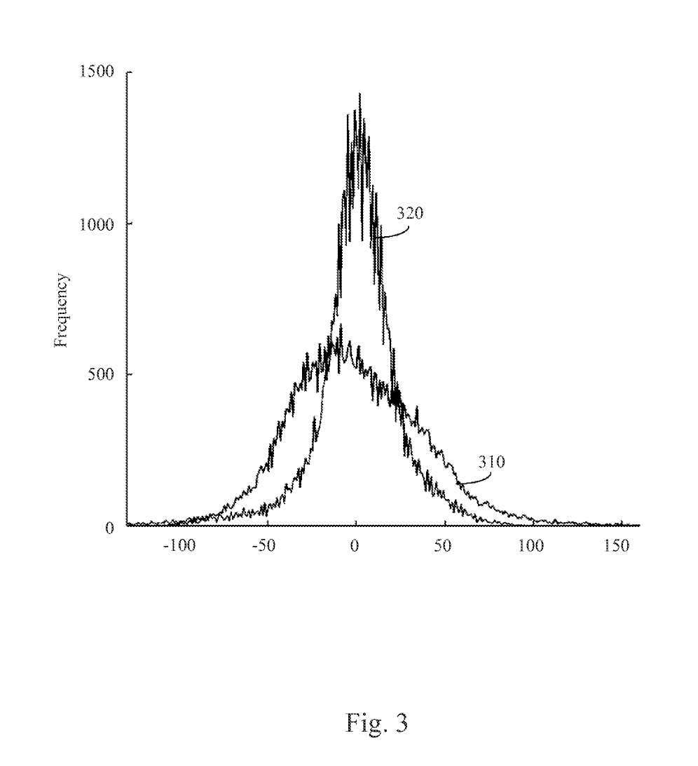

In RGB color model, PC1, PC2, and PC3 are three-dimensional PCA features because they use all red-, green-, and blue-channel information simultaneously. PCA is a linear transformation technique used to de-correlate data and maximize information content. FIG. 2 shows the skin lesion image pixels in the RGB color space, in which the pixel region 260 represents the lesion area, the pixel region 250 represents normal skin around the lesion area. The PCA technique basically analyzes an image's red, green, and blue values to obtain a new coordinate system (see axes 1-3 in FIG. 2; the first, second, and third principal axes 210, 220 and 230 are also called axes 1, 2, and 3 respectively for short), such that the greatest variance, known as the first principal component (PC1), lies on the first axis; the second principal component (PC2) is the greatest variance in a direction orthogonal to the first axis; and the third (PC3) is orthogonal to the first and second axes. Alternatively, the principal components PC1, PC2, PC3 can also be estimated by projecting every pixel's RGB values onto the three principal axes to form individual histograms for computing the corresponding variances (FIGS. 3-8). In this invention, the processor 110 computes all principal components for both the lesion region and the lesion plus some neighboring normal skin.

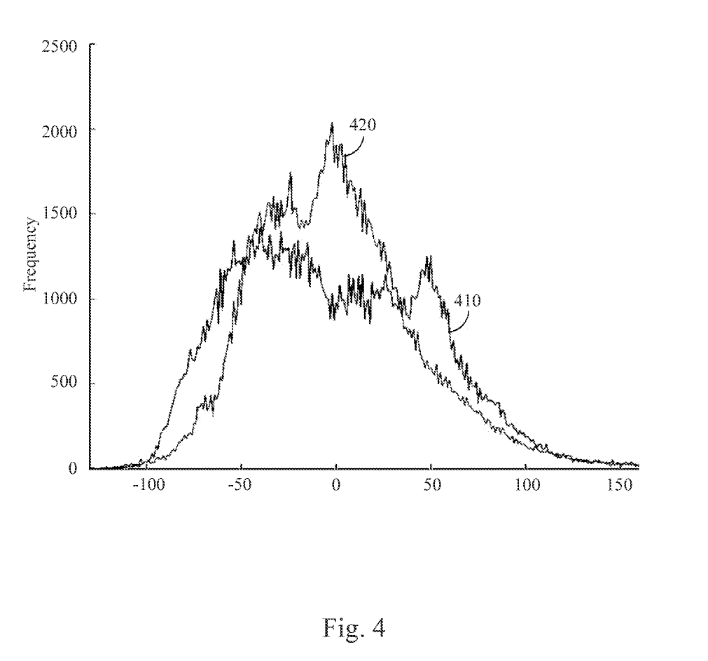

FIG. 3 shows a histogram of a benign skin tumor and a histogram of a normal skin portion surrounding the benign skin tumor according to one exemplary embodiment of the present disclosure, in which the curve 310 shows a histogram of a pixel projection of a lesion region on the first axis 210 (shown in FIG. 2), and the curve 320 shows a histogram of a pixel projection of a normal skin portion region on the first axis 210 (shown in FIG. 2). FIG. 4 shows a histogram of a malignant skin tumor and a histogram of a normal skin portion surrounding the malignant skin tumor according to another exemplary embodiment of the present disclosure, in which the curve 410 shows a histogram of a pixel projection of a lesion region on the first axis 210 (shown in FIG. 2), and the curve 420 shows a histogram of a pixel projection of a normal skin portion region on the first axis 210 (shown in FIG. 2).

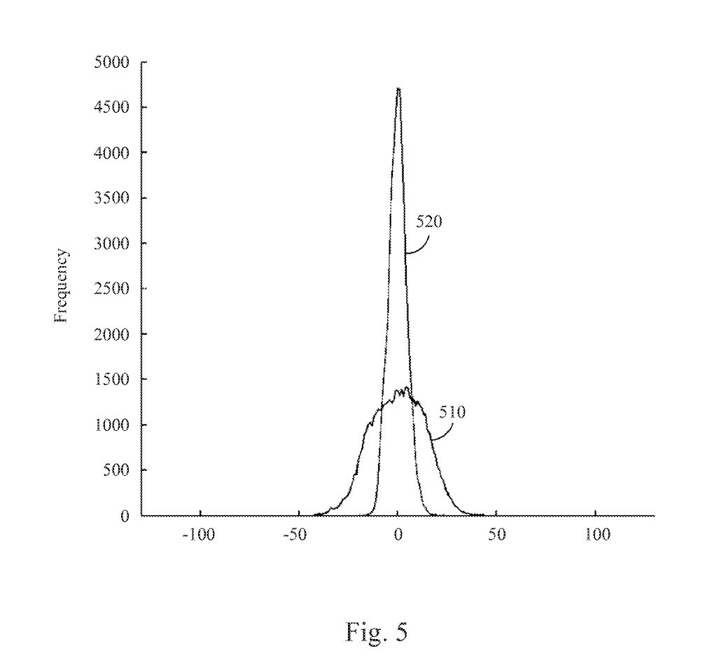

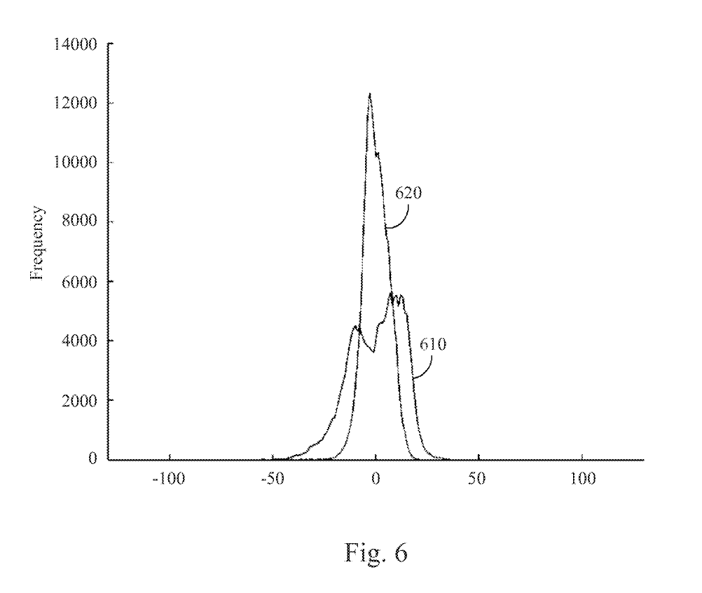

FIG. 5 shows a histogram of a benign skin tumor and a histogram of a normal skin portion surrounding the benign skin tumor according to one exemplary embodiment of the present disclosure, in which the curve 510 shows a histogram of a pixel projection of a lesion region on the second axis 220 (shown in FIG. 2), and the curve 520 shows a histogram of a pixel projection of a normal skin portion region on the second axis 220 (shown in FIG. 2). FIG. 6 shows a histogram of a malignant skin tumor and a histogram of a normal skin portion surrounding the malignant skin tumor according to another exemplary embodiment of the present disclosure, in which the curve 610 shows a histogram of a pixel projection of a lesion region on the second axis 220 (shown in FIG. 2), and the curve 620 shows a histogram of a pixel projection of a normal skin portion region on the second axis 220 (shown in FIG. 2).

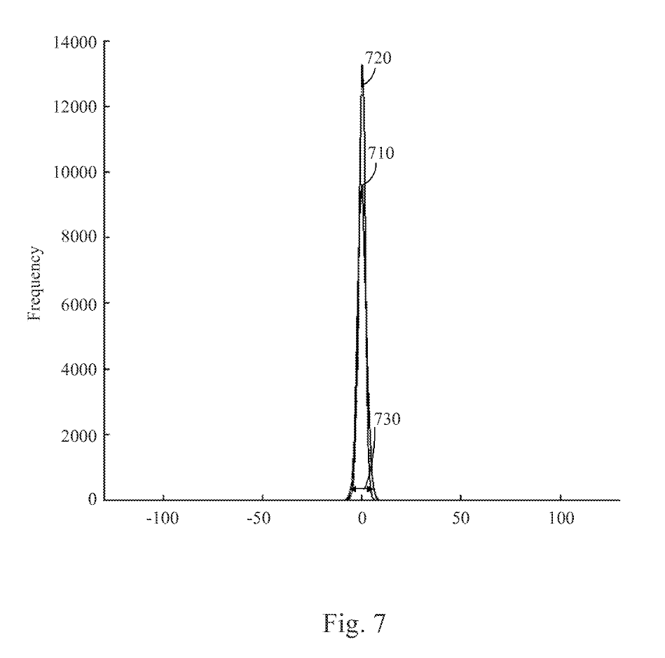

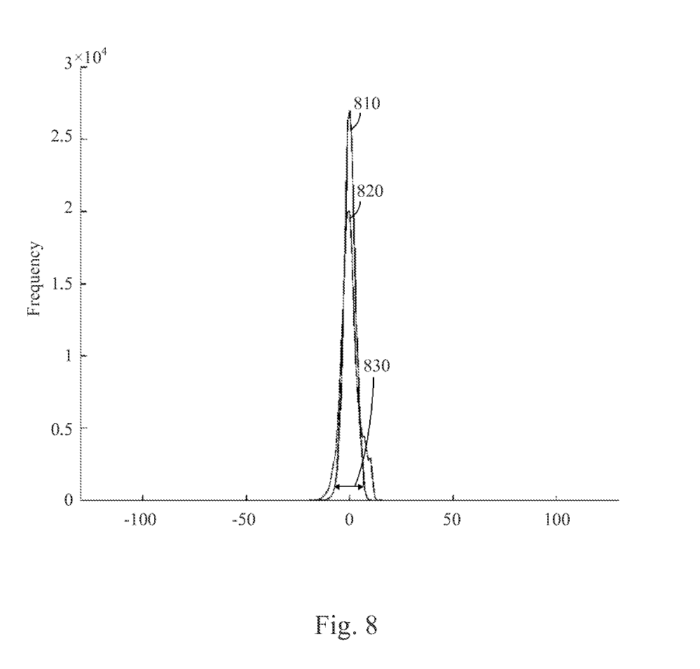

FIG. 7 shows a histogram of a benign skin tumor and a histogram of a normal skin portion surrounding the benign skin tumor according to one exemplary embodiment of the present disclosure, in which the curve 710 shows a histogram of a pixel projection of a lesion region on the third axis 230 (shown in FIG. 2), and the curve 720 shows a histogram of a pixel projection of a normal skin portion region on the third axis 230 (shown in FIG. 2). FIG. 8 shows a histogram of a malignant skin tumor and a histogram of a normal skin portion surrounding the malignant skin tumor according to another exemplary embodiment of the present disclosure, in which curve 810 shows a histogram of a pixel projection of a lesion region on the third axis 230 (shown in FIG. 2), and the curve 820 shows a histogram of a pixel projection of a normal skin portion region on the third axis 230 (shown in FIG. 2). Since a malignant lesion is more likely to have higher variegated colors, it has a wider histogram profile (830 in FIG. 8) on the third axis (shown in FIG. 2) than a benign lesion (730 in FIG. 7). Wider histograms on the third axis for malignant lesions mean that malignant lesions have higher PC3 than benign ones.

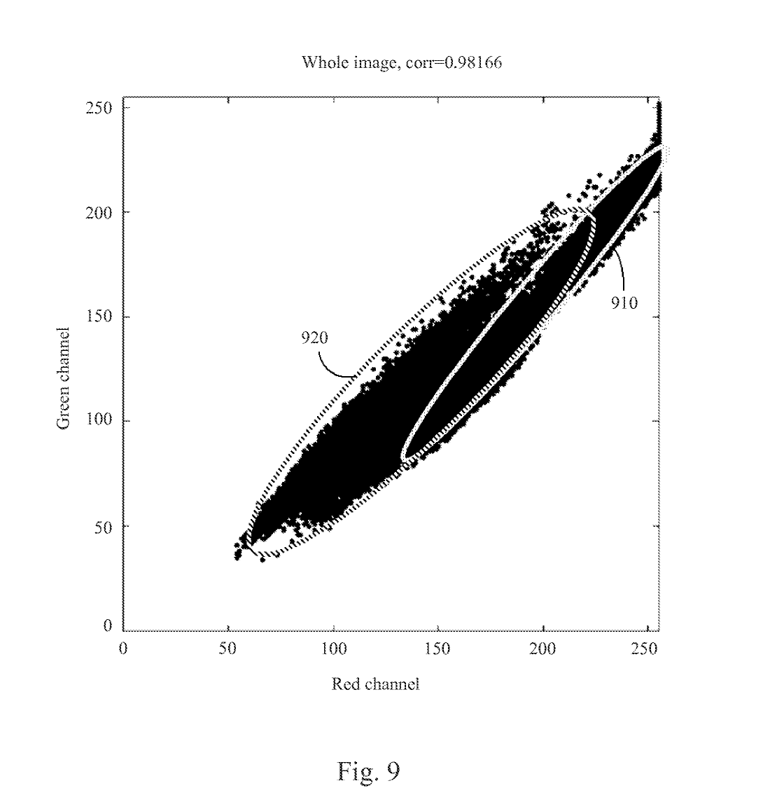

The color feature extraction also extracts two-dimensional color parameters by using the Pearson product-moment correlation coefficient. The correlation coefficient (e.g., Corr( )) is a measure of the linear dependence of two variables. FIG. 9 illustrates the correlation of red-, green-channel pixel values for a skin lesion image. Six correlation coefficients (red-green Corr (RG), green-blue Corr (GB), blue-red Corr (BR), red-brightness Corr (RX), green-brightness Corr (GX), blue-brightness Corr (BX)) are computed for both the lesion region and the lesion plus some neighboring normal skin pixels.

In FIG. 9, if the correlation coefficient (Corr) is higher, the distribution of the pixel is formed in a straight-line as a portion 910, in which the correlation coefficient (Corr)=0.95754 that represents normal skin with lower variegated colors, and it is noted that an absolute maximum value of the correlation coefficient is 1. If the correlation coefficient (Corr) is lower, the distribution of the pixel is spread as the other portion 920, in which the correlation coefficient (Corr)=0.86721 that represents lesion skin with higher variegated colors, and it is noted that an absolute minimum value of the correlation coefficient is 0.

It should be noted that other color features could be added into our invented system to improve the color variegation estimation.

Above machine learning algorithm (such as support vector machine (SVM) and other modern statistical learning methods) can learn the optimal way to combine the various color information to quantize the color variegation from the color features extraction.

Alternatively, it is possible to use our system by training the system to evaluate the malignancy of skin lesion using the color features implicitly (without deriving the color variegation feature explicitly).

In conclusion, the present disclosure provides an effective CADx system that has performance similar to that of the dermatologists at our institute and that classifies both melanocytic and non-melanocytic skin lesions by utilizing conventional digital macro-photographs. Through advanced feature selection and SVM analysis, we also found that the new color correlation and PCA features significantly improved CADx applications for skin cancer.

Although various embodiments of the invention have been described above with a certain degree of particularity, or with reference to one or more individual embodiments, they are not limiting to the scope of the present disclosure. Those with ordinary skill in the art could make numerous alterations to the disclosed embodiments without departing from the spirit or scope of this invention. Accordingly, the protection scope of the present disclosure shall be defined by the accompany claims.

What is claimed is:

1. A computer-aided diagnosis system, comprising:
a processor capable of executing one or more computer executable instructions;
a memory comprising a computer program executable by the processor, the computer program which, when executed by the processor:
performing a principal component analysis to acquire an effect of light and shade portions from a color image of skin to serves as a first principal component.

2. The computer-aided diagnosis system of claim 1, wherein the principal component analysis further analyzes a second principal component and a third principal component, and the second and third principal components contain color variability.

3. The computer-aided diagnosis system of claim 2, wherein the third principal component is correlated with a skin cancer and serves as a main indicator of variegated colors.

4. The computer-aided diagnosis system of claim 3, wherein the processor acquires a two-dimensional correlation coefficient from the color images, the two-dimensional correlation coefficient is different from the principal component analysis and is computed by machine learning to enhance an accuracy of malignancy index of the variegated colors.

5. The computer-aided diagnosis system of claim 4, wherein the processor acquires one of more one-dimensional statistical parameters from the color images, the one-dimensional statistical parameters including a variance parameter, an entropy parameter and a skewness parameter are different from the principal component analysis and is computed by machine learning to enhance the accuracy of the malignancy index of the variegated colors.

6. The computer-aided diagnosis system of claim 5, further comprising:
an image-capturing device configured to capture the color image of the skin.

7. The computer-aided diagnosis system of claim 6, wherein the image-capturing device is a camera.

8. The computer-aided diagnosis system of claim 6, wherein the color image includes a lesion and a normal skin portion surrounding the lesion to improve a stability of the malignancy index of the variegated colors.

9. The computer-aided diagnosis system of claim 5, wherein the processor is configured to diagnose a skin cancer based on the first, second and third principal components, the two-dimensional correlation coefficient and one-dimensional statistical parameter

10. The computer-aided diagnosis system of claim 9, wherein the principal component analysis is applied in a RGB color model.

11. A computer-aided diagnosis method, comprising:
performing a principal component analysis to acquire an effect of light and shade portions from a color image of skin to serves as a first principal component.

12. The computer-aided diagnosis method of claim 11, wherein the principal component analysis further analyzes a second principal component and a third principal component, and the second and third principal components contain color variability.

13. The computer-aided diagnosis method of claim 12, wherein the third principal component is correlated with a skin cancer and serves as a main indicator of variegated colors.

14. The computer-aided diagnosis method of claim 13, further comprising:
acquiring a two-dimensional correlation coefficient from the color images, the two-dimensional correlation coefficient is different from the principal component analysis and is computed by machine learning to enhance an accuracy of malignancy index of the variegated colors.

15. The computer-aided diagnosis method of claim 14, further comprising:
acquiring one of more one-dimensional statistical parameters from the color images, the one-dimensional statistical parameters including a variance parameter, an entropy parameter and a skewness parameter are different from the principal component analysis and is computed by machine learning to enhance the accuracy of the malignancy index of the variegated colors.

16. The computer-aided diagnosis method of claim 15, further comprising:
capturing the color image of the skin by an image-capturing device.

17. The computer-aided diagnosis method of claim 16, wherein the image-capturing device is a camera.

18. The computer-aided diagnosis method of claim 16, wherein the color image includes a lesion and a normal skin portion surrounding the lesion to improve a stability of the malignancy index of the variegated colors.

19. The computer-aided diagnosis method of claim 15, further comprising:
diagnosing a skin cancer based on the first, second and third principal components, the two-dimensional correlation coefficient and one-dimensional statistical parameter.

20. The computer-aided diagnosis method of claim 19, wherein the principal component analysis is applied in a RGB color model.
